(19) United States
(12) Patent Application Publication
(10) Pub. No.: US 2003/0113362 A1
(43) Pub. Date: Jun. 19, 2003

(54) Unique ascorbic acid derivatives with essential amino acids, nonessential amino acid and amino acids that do not occur in protein: the process for their preparation and use in research, medicine, physiology, pharmacology, nutrition, pharmaceuticals, and for cosmetic, commercial and industrial applications

(76) Inventors: Waheed M. Roomi, Sunnyvale, CA (US); Shrirang Netke, Cupertino, CA (US); Vadim Ivanov, Castro Valley, CA (US); Aleksandra Niedzwiecki, San Jose, CA (US); Matthias Rath, San Jose, CA (US)

Correspondence Address: Ali Kamarei, Esq., 280 Colorado Avenue, Palo Alto, CA, 94301, US

(21) Appl. No.: 10/226,546

(22) Filed: Aug. 23, 2002

Related U.S. Application Data
(60) Provisional application No. 60/314,848, filed on Aug. 24, 2001.

(51) Int. Cl. A61K 31/4178; C 07D 4 5/02; A61K 47/00

(52) U.S. Cl. 424/439; 548/315.4; 548/465; 548/517; 549/317

(57) ABSTRACT

A biochemical compound is described comprising ascorbic acid and at least one essential aminoacid, wherein the ascorbic acid is covalently bound to the aminoacid. The present invention provides a method of use of a biochemical compound where ascorbate molecules are covalently bound to essential amino acids, nonessential amino acids and amino acids that do not occur in protein to prevent oxidation.

### CLAIM OF PRIORITY

Applicant hereby claims priority to U.S. Provisional Application No. 60/314,848, filed on Aug. 24, 2001.

This invention was not made by an agency of the United States Government or under a contract with an agency of the United States Government.

### FIELD OF THE INVENTION

The invention relates to the unique ascorbic acid compounds with essential amino acid, nonessential amino acid and other amino acids that do not occur in protein, the process for their preparation and the method of use in research, medicine, physiology, pharmacology, pharmaceuticals, nutrition and for cosmetic, commercial and industrial application.

### BACKGROUND OF THE INVENTION

The feeding of essential amino acids is warranted under conditions in which there is dietary insufficiency of these amino acids. Such acids are arginine, methionine, lysine, phenylalanine, tyrosine, valine, leucine, isoleucine, tryptophan, and threonine. Feeding of these amino acids along with nonessential amino acids which include glycine, alanine, serine, cysteine, aspartic acid, asparagines, glutamic, glutamine, tyrosine, proline, hydroxyproline and amino acids that do not occur in protein such as ornithine, citruline and beta alanine.

Feeding of these amino acids along with nonessential amino acids could be desirable in tissue level starvations obtained in diabetes and metabolic stress conditions resulting from injury to the body like surgery, infections or burns. Intravenous administration of combinations of essential and nonessential amino acids is resorted to under certain conditions of starvation. In this all these situations feeding of compounds of ascorbic acid and amino acids would be beneficial. This is in continuation of our previous invention on the novel synthesis of ascorbic acid compounds with lysine and its derivatives and/or proline and its derivatives.

Ascorbic acid is a ubiquitous compound essential for the maintenance and preservation of several species. In human beings deprived of ascorbic acid, the deficiency disease scurvy develops which can be life threatening. Ascorbic acid is probably the most effective, efficient and least toxic antioxidant. It is a water soluble, chain-breaking antioxidant, that acts as scavenger for harmful radicals like superoxide, hydroxyl and singlet oxygen that are produced during normal cellular metabolism. Ascorbic acid is superior to other water soluble and lipid soluble antioxidants. It also protects DNA, enzyme, protein and lipids from oxidative damage and thereby prevents aging, coronary heart diseases, cataract formation, degenerative diseases and cancer. Oxygen radicals have been implicated in initiation and post-initiation stages of carcinogenesis, and in invasion and metastatic processes.

Ascorbic acid has several physiological functions. It is essential for collagen synthesis, proteoglygans and various components of extra cellular matrix (ECM). It also helps maintain various enzymes in their reduced forms. Ascorbic acid is involved in the hydroxylation of lysine and proline for which ascorbic acid functions as cofactor. Lysine and proline are principal components of tendons, ligaments, skin, bone, teeth, cartilage, heart valves, cornea, eye lens and ground substances between cells fibers. Any deficiency of ascorbic results in impaired collagen formation which leads to tissue weakness and eventually, scurvy. Cellular medicine considers ascorbic acid tile most important natural substances indispensable for maintenance of health at the cellular level. Deficiency of ascorbic acid in humans may lead to various diseases. In addition, ascorbic acid participates in the biosynthesis of carnitine and neuroendocrine peptides.

Ascorbic acid has several reactive hydroxy groups that can be used for the synthesis of a number of derivatives. Many substituted compounds at 2-, 3-, 5- and 6-positions have been synthesized. L-ascorbate 2-sulphate is stored in fish and some shrimp. It has ascorbic acid activity for fish such as trout, salmon and catfish. It is 20 times more stable than ascorbic acid. Hence, it has been used in the formulation of feeds. L-ascorbate 2-phosphate is more stable in air than ascorbic acid. This compound is used as source of ascorbic in guinea pigs and rhesus monkeys. L-ascorbyl 6-palmitate, a synthetic lipophilic ascorbic acid derivative, is an effective preservative in foods and pharmaceuticals. In recent years there has been a growing interest in the therapeutic application of Lascorbic acid and its derivatives as an anti-cancer agent.

### SUMMARY OF INVENTION

The present invention focuses on the synthesis of various compounds of ascorbic acid with essential amino acid, nonessential amino acid and amino acid that do not occur in protein. These compounds could be used in research medicine, physiology, pharmacology, pharmaceuticals, nutrition and cosmetic, commercial and industrial application.

The overall objective of this invention is to synthesize ascorbic acid derivatives with essential, nonessential and amino acids that do not occur in protein. The synthesis is carried using L-ascorbic acid and the amino acids. For this purpose, the CH₂OH of ascorbic acid at 6-position and carboxyl groups of amino acids is utilized.’

These new biochemical compounds can provide additional biological effects superior to its individual compounds. Thus, a biochemical synthesis of these compounds in which the amino acids are covalently bound to ascorbic acid is preferable to a simple physical mixture of the amino acids with ascorbic acid.

Such unexpected superior biological effects include increased biological stability of these molecules, enhanced absorption by various biological cell compartments and greater biological efficacy. Such compounds can facilitate and enhance the assimilation of other nutritional components from foods resulting in improved nutritional status of individuals.

These novel compounds have applications in a variety of areas including but not limited to nutrition, medicine, and pharmacology.

### BRIEF DESCRIPTION OF THE DRAWINGS

FIG. 1 shows the structure of various essential amino acids.

FIG. 2 shows the structure of various nonessential amino acids.

FIG. 3 shows the structure of various amino acids that do not occur in proteins.

FIG. 4 shows the scheme for the synthesis of ascorbic acid derivatives with essential amino acids. A typical example is that of ascorbyl 6-phenyalanine.

FIG. 5 shows the scheme for the synthesis of ascorbic acid derivatives with nonessential amino acids. A typical example is that of ascorbyl 6-glycine.

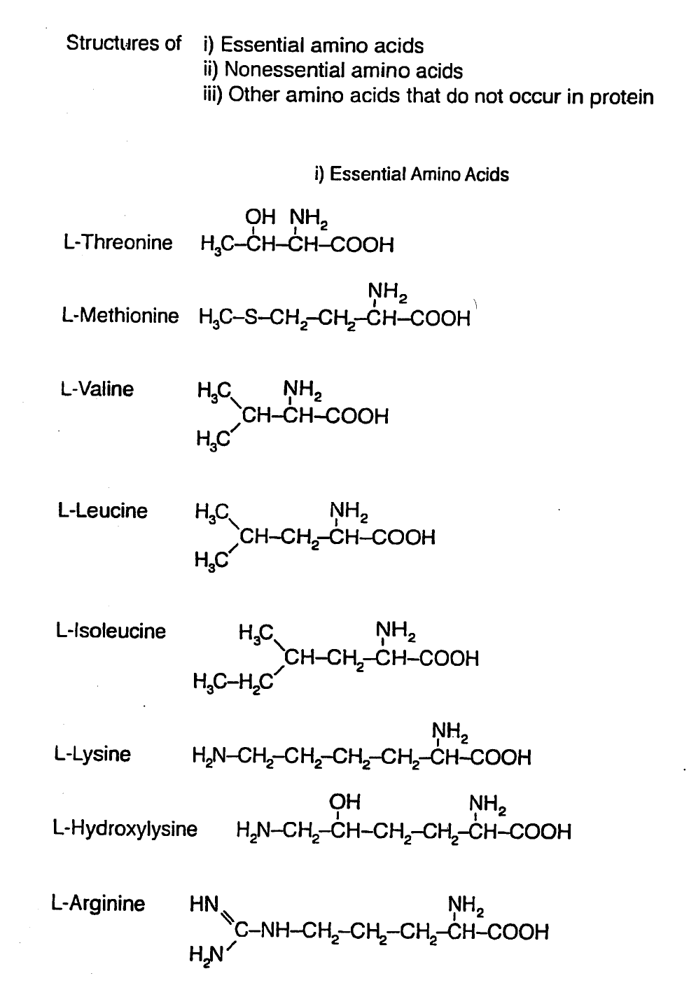

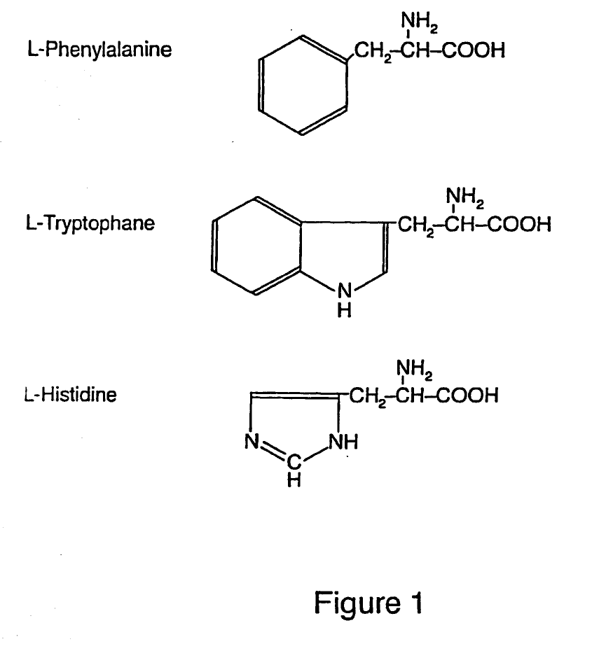

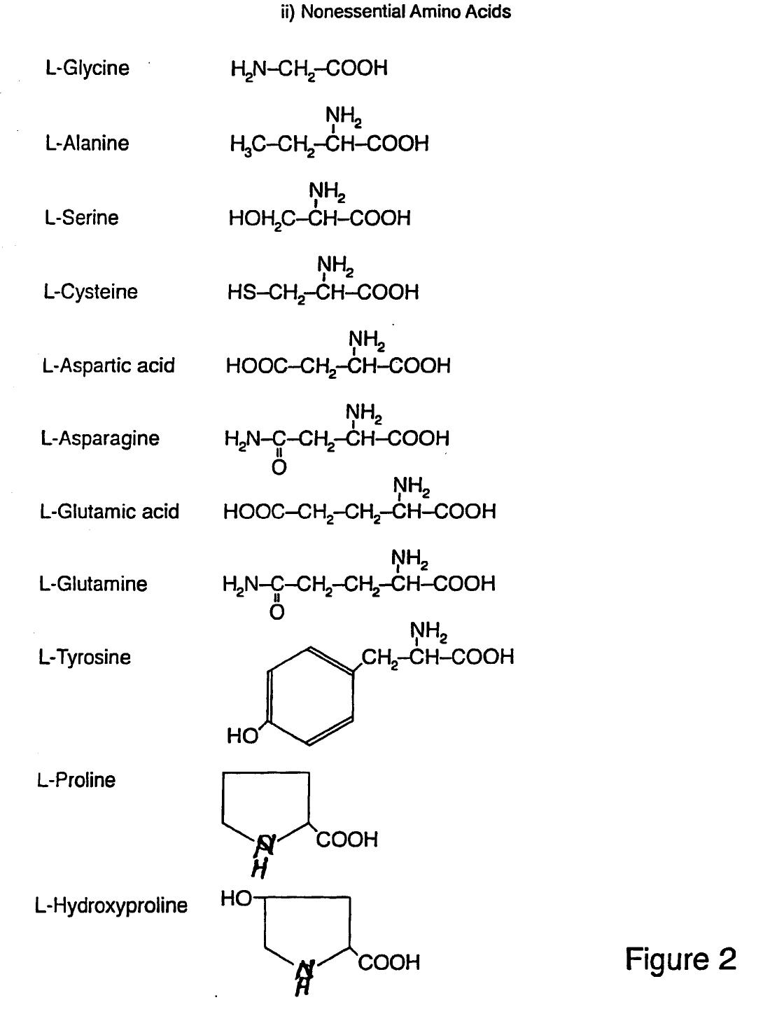

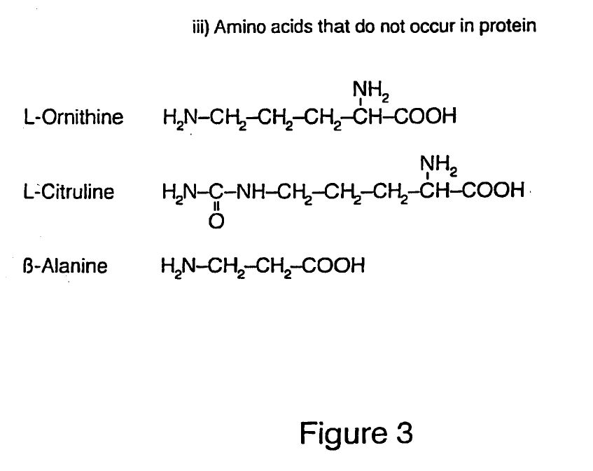

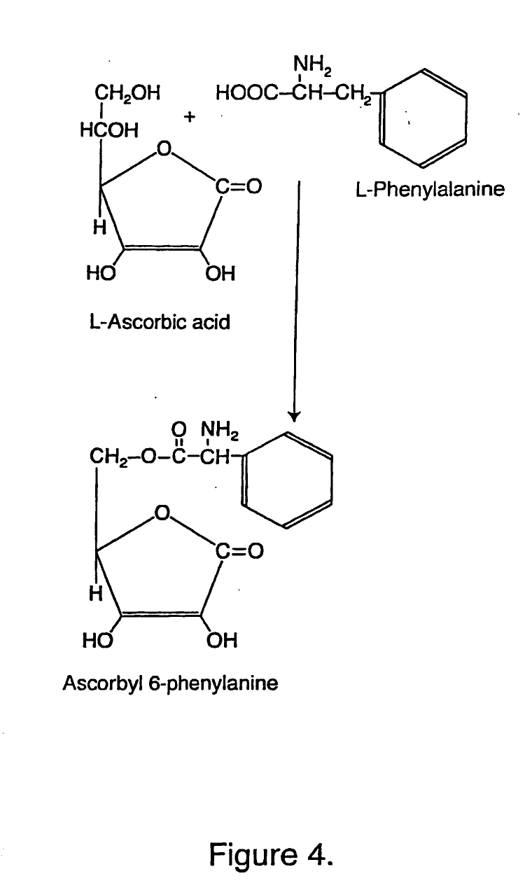

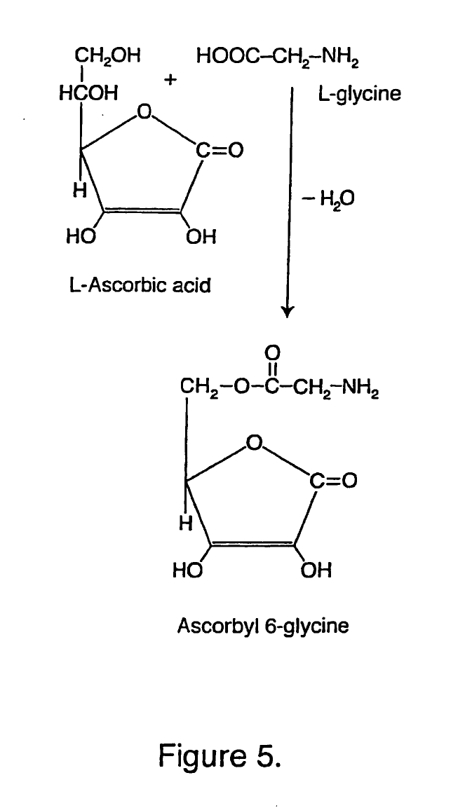

### DETAILED DESCRIPTION OF THE INVENTION

The present invention provides the synthesis of biochemical compounds where ascorbate molecules are covalently bound to essential amino acids, nonessential amino acids and amino acids that do not occur in protein.

The present invention provides the synthesis of a biochemical compound where one essential amino acid is bond to ascorbic acid in C-6 position.

The present invention provides the synthesis of a biochemical compound where one essential amino acid is bond to ascorbic acid in C-2 position.

The present invention provides the synthesis of a biochemical compound where one essential amino acid is bond to ascorbic acid in C-6 and C-2 positions.

The present invention provides the synthesis of a biochemical compound where one non essential amino acid is bond to ascorbic acid in C-6 position.

The present invention provides the synthesis of a biochemical compound where one nonessential amino acid is bond to ascorbic acid in C-2 position.

The present invention provides the synthesis of a biochemical compound where on nonessential amino acid is bond to ascorbic acid in C-6 and C-2 positions.

The present invention provides the synthesis of a biochemical compound where an amino acid that do not occur in protein is bond to ascorbic acid at C-6 position.

The present invention provides the synthesis of a biochemical compound where an amino acid that do not occur in protein is bond to C-2 position.

The present invention provides the synthesis of a biochemical compound where an amino acid that do not occur in protein is bond to C-6 and C-2 position.

The present invention provides the synthesis of biochemical compound where two or more essential amino acid are bond to ascorbic acid at C-6 position.

The present invention provides the synthesis of a biochemical compound where two or more essential amino acids arc bond to ascorbic acid at C-2 position.

The present invention provides the synthesis of a biochemical compound where two or more essential amino acids are bond to ascorbic acid at C-6 and C-2 positions.

The present invention provides the synthesis of a biochemical compound where two or more nonessential amino acid are bond ascorbic acid at C-6 position.

The present invention provides the synthesis of a biochemical compound where two or more nonessential amino acids are bond to ascorbic acid at C-2 position.

The present invention provides the synthesis of a biochemical compound where two or more nonessential amino acids are to ascorbic acid at C-6 and C-2 position.

The present invention provides the synthesis of biochemical compound where two or more amino acids that do not occur in protein are bond to ascorbic acid at C-6 position.

The present invention provides the synthesis of a biochemical compound where two or more amino acids that do not occur in protein are bond to ascorbic acid at C-2 position.

The present invention provides the synthesis of a biochemical compound where two or more amino acids that do not occur in protein are bond to ascorbic acid at C-6 and C-2 positions.

The present invention provides the synthesis of a biochemical compound where one or more essential amino acids are bond to ascorbic acid at C-6 position and one or more. nonessential amino acids are bond to 2-position.

The present invention provides the synthesis of a biochemical compound where one or more essential amino acids are bond to ascorbic acid at C-6 position and one or more amino acids that do not occur in protein arc bond to C-2 position.

The present invention provides the synthesis of a biochemical compound where one or more nonessential amino acids are bond to ascorbic acid at C-6 position and one or more essential amino acids are bond to C-2 position.

The present invention provides the synthesis of a biochemical compound where one or more nonessential amino acids are bond to ascorbic acid at C-6 position and one or more amino acids that do not occur in protein are bond to C-2 position.

The present invention provides the synthesis of a biochemical compound where one or more amino acids that do not occur in protein are bond to ascorbic acid at C-6 position and one or more essential amino acids are bond to C-2 position.

The present invention provides the synthesis of a biochemical compound where one or more amino acids that do not occur in protein are bond to ascorbic acid at C-6 position and one or more nonessential amino acids are bond to C-2 position.

The present invention provides a method of use of a biochemical compound where ascorbate molecules are covalently bound to essential amino acids, nonessential amino acids and amino acids that do not occur in protein to prevent oxidation.

The present invention provides a method of use of a biochemical compound where ascorbate molecules are covalently bound to essential amino acids, nonessential amino acids and amino acids that do not occur in protein to prevent and/or retard aging of organic and inorganic materials.

The present invention provides a method of use of a biochemical compound where ascorbate molecules are covalently bound to essential amino acids, nonessential amino acids and amino acids that do not occur in protein to be used in preventive and therapeutic medicine.

The present invention provides a method of use of a biochemical compound where ascorbate molecules are covalently bound to essential amino acids, nonessential amino acids and amino acids that do not occur in protein to stabilize connective tissue and prevent the degradation of extracellular matrix.

The present invention provides a method of use of a biochemical compound where ascorbate molecules are covalently bound to essential amino acids, nonessential amino acids and amino acids that do not occur in protein to be used in the identification, development and use of pharmaceutical compounds and their preparations.

The present invention provides a method of use of a biochemical compound where ascorbate molecules are covalently bound to essential amino acids, nonessential amino acids and amino acids that do not occur in protein to be used in foods, beverages, and nutritional supplements.

The present invention provides a method of use of a biochemical compound where ascorbate molecules are covalently bound to essential amino acids, nonessential amino acids and amino acids that to prevent do not occur in protein to be used in chemical synthesis process.

The present invention provides a method of use of a biochemical compound where ascorbate molecules are covalently bound to essential amino acids, nonessential amino acids and amino acids that do not occur in protein to be used in the industry process.

The present invention provides a method of use of a biochemical compound where ascorbate molecules are covalently bound to essential amino acids, nonessential amino acids and amino acids that do not occur in protein to be used in the industry producing cosmetic products.

The present invention is by no means restricted to these specific embodiments and not to be limited in scope by specific examples described herein. Various modifications of the present invention to those described herein will become apparent to those skilled in the art from the foregoing description and accompanying figures. Such modifications are intended to fall within the scope of the present invention.

What is claimed is:

1. A biochemical compound, comprising:
a. ascorbic acid and
b. at least one essential aminoacid, wherein the ascorbic acid is covalently bound to the aminoacid.

2. The biochemical composition of claim 1 wherein one essential amino acid is bound to ascorbic acid in the C-2 position.

3. The biochemical composition of claim 1 wherein an essential amino acid is bound to ascorbic acid in C-6 and C-2 positions.

4. The biochemical composition of claim 1 wherein one non essential amino acid is bound to ascorbic acid in C-6 position.

5. The biochemical composition of claim 1 wherein one nonessential amino acid is bound to ascorbic acid in C-2 position.

6. The biochemical composition of claim 1 wherein an nonessential amino acid is bound to ascorbic acid in C-6 and C-2 positions.

7. The biochemical composition of claim 1 wherein an amino acid that do not occur in protein is bound to ascorbic acid at C-6 position.

8. The biochemical composition of claim 1 wherein an amino acid that do not occur in protein is bound to C-2 position.

9. The biochemical composition of claim 1 wherein an amino acid that do not occur in protein is bound to C-6 and C-2 position.

10. The biochemical composition of claim 1 wherein two or more essential amino acid are bound to ascorbic acid at C-6 position.

11. The biochemical composition of claim 1 wherein two or more essential amino acids arc bound to ascorbic acid at C-2 position.

12. The biochemical composition of claim 1 wherein two or more essential amino acids are bound to ascorbic acid at C-6 and C-2 positions.

13. The biochemical composition of claim 1 wherein two or more nonessential amino acid are bound ascorbic acid at C-6 position.

14. The biochemical composition of claim 1 wherein two or more nonessential amino acids are bound to ascorbic acid at C-2 position.

15. The biochemical composition of claim 1 wherein two or more nonessential amino acids are to ascorbic acid at C-6 and C-2 position.

16. The biochemical composition of claim 1 wherein two or more amino acids that do not occur in protein are bound to ascorbic acid at C-6 position.

17. The biochemical composition of claim 1 wherein two or more amino acids that do not occur in protein are bound to ascorbic acid at C-2 position.

18. The biochemical composition of claim 1 wherein two or more amino acids that do not occur in protein are bound to ascorbic acid at C-6 and C-2 positions.

19. The biochemical composition of claim 1 wherein one or more essential amino acids are bound to ascorbic acid at C-6 position and one or more nonessential amino acids are bound to 2-position.

20. The biochemical composition of claim 1 wherein one or more essential amino acids are bound to ascorbic acid at C-6 position and one or more amino acids that do not occur in protein arc bound to C-2 position.

21. The biochemical composition of claim 1 wherein one or more nonessential amino acids are bound to ascorbic acid at C-6 position and one or more essential amino acids are bound to C-2 position.

22. The biochemical composition of claim 1 wherein one or more nonessential amino acids are bound to ascorbic acid at C-6 position and one or more amino acids that do not occur in protein are bound to C-2 position.

23. The biochemical composition of claim 1 wherein one or more amino acids that do not occur in protein are bound to ascorbic acid at C-6 position and one or more essential amino acids are bound to C-2 position.

24. The biochemical composition of claim 1 wherein one or more amino acids that do not occur in protein are bound to ascorbic acid at C-6 position and one or more nonessential amino acids are bound to C-2 position.

25. The present invention provides a method of use of a biochemical compound where ascorbate molecules are covalently bound to essential amino acids, nonessential amino acids and amino acids that do not occur in protein to prevent oxidation.

26. The biochemical composition of claim 1 wherein ascorbate molecules are covalently bound to essential amino acids, nonessential amino acids and amino acids that do not occur in protein to prevent and/or retard aging of organic and inorganic materials.

27. The biochemical composition of claim 1 wherein ascorbate molecules are covalently bound to nonessential amino acids and amino acids that do not occur in protein to be used in preventive and therapeutic medicine.

28. The biochemical composition of claim 1 wherein ascorbate molecules are covalently bound to essential amino acids, nonessential amino acids and amino acids that do not occur in protein to stabilize connective tissue and prevent the degradation of extracellular matrix.

29. The biochemical composition of claim 1 wherein the ascorbate molecules are covalently bound nonessential amino acids and amino acids that do not occur in protein to be used in foods, beverages, and nutritional supplements.

30. The biochemical composition of claim 1 wherein the ascorbate molecules are covalently bound to nonessential amino acids and amino acids that to prevent do not occur in protein to be used in chemical synthesis process.

31. The biochemical composition of claim 1 wherein the ascorbate molecules are covalently bound to nonessential amino acids and amino acids that do not occur in protein to be used in the industry process.
